(19) United States
(12) Patent Application Publication
(10) Pub. No.: US 2013/0216499 A1
(43) Pub. Date: Aug. 22, 2013

(54) COMPOSITIONS OF RECOMBINANT HUMAN ENDOSTATIN ADENOVIRUS INJECTIONS AND METHODS OF PRODUCTION

(71) Applicant: Wenlin Huang, Chadds Ford, PA (US)

(72) Inventor: Wenlin Huang, Chadds Ford, PA (US)

(21) Appl. No.: 13/762,185

(22) Filed: Feb. 7, 2013

Related U.S. Application Data
(60) Provisional application No. 61/599,994, filed on Feb. 17, 2012.

(51) Int. Cl. A61K 9/19; A61K 35/76

(52) U.S. Cl. CPC A61K 9/19; A61K 35/761
USPC 424/93.2

(57) ABSTRACT

The invention generally relates to compositions of and methods for production of recombinant adenoviruses that carry therapeutic genes. More particularly, the invention relates to lyophilized recombinant adenoviruses injection and its related production procedures, including production procedures for the recombinant adenovirus vectors (or other viral vectors) that carry the genes of human endostatins.

### PRIORITY CLAIMS AND RELATED PATENT APPLICATIONS

This application claims the benefit of priority from U.S. Provisional Application Ser. No. 61/599,994, filed on Feb. 17, 2012, the entire content of which is incorporated herein by reference in its entirety.

### TECHNICAL FIELD OF THE INVENTION

The invention generally relates to compositions of and methods for production of recombinant adenoviruses. More particularly, the invention relates to lyophilized recombinant adenoviruses injection and its related production procedures, including production procedures for the lyophilized recombinant adenovirus vectors that carry the genes of human endostatins.

### BACKGROUND OF THE INVENTION

In recent years, significant research efforts have been devoted to the development of genetic therapies for malignant tumors, including applying virus as a vector to the genetic therapies. Adenovirus vectors provide a number advantages, including high transduction efficiency, wide coverage of various kinds of hosts, capability to infect dividing and non-dividing cells, the large number of exogenous genes, ease at manufacture, and the viral genome cannot integrate into host chromosome.

Angiogenesis is the physiological process involving the growth of new blood vessels from pre-existing vessels. (See e.g., John S. Penn (11 Mar. 2008) Retinal and Choroidal Angiogenesis Springer, ISBN 9781402067792.) As a fundamental step in the transition of tumors from a primary state to a metastatic one, angiogenesis is a key step for the spread of a tumor, or metastasis. Endothelial cells are reported to be genetically more stable than cancer cells, which confer an advantage to targeting endothelial cells using antiangiogenic therapy.

Angiogenesis is a complicated, multistep process that includes degradation of the basement membrane, vascular endothelial cell proliferation, migration and differentiation. The process ultimately forms new blood vessels to provide oxygen and nutrition for the surrounding tissue. New blood vessel formation is determined by the balance between angiogenesis stimulatory factors and inhibitory factors in the microenvironment.

Endostatin is a naturally occurring antiangiogenic protein that is reported to inhibit the formation of neovascular that provides nutrition and oxygen to tumors. It was first discovered in the Children's Hospital Boston laboratory of Judah Folkman. As an endogenous angiogenesis inhibitor, endostatin may interfere with the pro-angiogenic action of growth factors such as basic fibroblast growth factor (bFGF/FGF-2) and vascular endothelial growth factor (VEGF). (Folkman, J. (2006) Exp. Cell. Res. 312 (5): 594-607.)

Endostatin was first found secreted in the media of non-metastasizing mouse cells from a hemangioendothelioma cell line and was subsequently found in human. Endostatin was reported to play a role in extracellular matrix in suppression of neoangiogenesis. (O'Reilly, et al. (1997) Cell 88: 277-85; Standker, et al. (1997) FEBS Lett. 420: 129-33.)

Endostatin has been identified as a C-terminal fragment of Collagen type 18. Endostatin has a short half-life and its therapeutic effect is in dose-dependent manner Without wishing to be bound by the theories, endostatin represses cell cycle control and anti-apoptosis genes in proliferating endothelial cells, resulting in cell death. (Shichiri, et al. (2001). FASEB J 15: 1044-53.) Endostatin blocks pro-angiogenic gene expression controlled by c-Jun N terminal kinase (JNK) by interfering with TNFα activation of JNK. (Yin, et al. (2002) Mol. Ther. 5: 547-54.) It reduces the growth of new cells by inhibiting cyclin D1. As a result, cells arrest during G1 phase and enter apoptosis. (Dhanabal, et al. (1999) Biochem Biophys Res. Comm. 258: 345-52; Hanai, et al. (2002) J. Biol. Chem. 277: 16464-9.)

Endostatin has been reported to have several advantages in its use for cancer therapy. Endogenous endostatin has shown little or no resistance or toxicity in human compared to other cancer drugs. Endostatin has been estimated to affect about 12% of the human genome and offers a broad spectrum of potential activity as compared to single-molecule therapies.

Endostatin was first found secreted in the media of non-metastasizing mouse cells, and is known to be capable of inhibiting the growth and metastasizing of endothelial cells and subsequently leading to the death of the endothelial cells. However, endothelial cells tend to be unstable outside human bodies; therefore, it's difficult to produce endothelial cells in large quantities.

### SUMMARY OF THE INVENTION

The present invention is based, in part, on the novel approach to preparation of pharmaceutical compositions of recombinant adenoviruses, which involves lyophilization of recombinant adenoviruses in production procedures for the finished product.

In one aspect, the invention generally relates to pharmaceutical composition that includes: a lyophilized recombinant human endostatin with adenovirus; and a preserver composition comprising: a sugar, a polyol, an amino acid, and a salt, wherein the recombinant human endostatin with adenovirus comprises a human endostatin gene.

In another aspect, the invention generally relates to a method of preparing the pharmaceutical composition. The method includes: (1) mixing one or more excipients in proper amounts and purified water resulting in a solution; (2) filtering the resulting solution with a 0.22-μm filter to remove bacteria; (3) adding recombinant human endostatin with adenovirus while mixing and making the mixed solution a pH in a range from about 8.0 to about 8.6; (5) lyophilizing the mixed solution; (6) maintaining a vacuum under 3 Pa; and (7) storing the resulting lyophilized mixture.

### BRIEF DESCRIPTION OF THE DRAWINGS

FIG. 1 shows certain exemplary stability comparison curves between lyophilized and liquidized samples at 37° C.

### DETAILED DESCRIPTION OF THE INVENTION

The present invention is based, in part, on a unique approach to the production of pharmaceutical compositions of recombinant adenoviruses that involves lyophilizing recombinant adenoviruses. Pharmaceutical compositions of the invention, for example, recombinant adenoviruses injections, are safe, effective, stable and can be stored for a long period of time.

Human endostatin is an angiogenesis inhibitor that inhibits the proliferation of vascular endothelial cells to achieve its purpose of inhibiting angiogenesis.

Adenoviruses are viruses that carry their genetic material in the form of double-stranded DNA. When adenoviruses infect a host cell, they introduce their DNA molecule into the host. The genetic material of the adenoviruses is not incorporated into the host cell's genetic material. The DNA molecule is left free in the nucleus of the host cell, and the instructions in this extra DNA molecule are transcribed just like any other gene. The only difference is that these extra genes are not replicated when the cell is about to undergo cell division so the descendants of that cell will not have the extra gene. As a result and generally speaking, treatment with the adenovirus will require re-administration in a growing cell population.

Here, the recombinant human endostatin adenovirus refers to the introduction of an improved type V adenovirus gene into human endostatin genes. The recombinant adenovirus serves as the vector to introduce human endostatin genes into the host cell gene structure, and yields human endostatin protein via gene expression. Human endostatin adenovirus of the present invention is a replication-defective, recombinant oncolytic adenovirus encoding human endostatin with potential antineoplastic activity. Upon intratumoral administration, the adenovirus infects and replicates in tumor cells. The expressed endostatin may inhibit endothelial cell proliferation and angiogenesis that may result in a reduction of tumor growth.

Adenoviruses can travel directly to where a tumor grows and starts to absorb the steady supply of endostatins from within the body and helps with suppressing the proliferation of endothelial cells and angiogenesis of the tumor by blocking the blood supply to the tumorous tissue thereby inhibiting the growth of the tumor, which eventually leads to the death of the tumor. Recombinant human endostatin adenoviruses has been approved by SFDA to start Phase II clinical trail.

Adenoviruses are relatively stable in protein preparation and have good half-life, activity, as well as reasonable cost for production. When in storage, however, adenoviruses have low stability and are very sensitive to freeze-thaw cycles. Therefore, in order to maintain bio-stability, adenoviruses are usually preserved in a buffer solution containing about 10% of glycerine and are stored under a temperature of −80° C. Such preservation method tends to involve high cost because dry ice is typically required during long-distance transportation. Additionally, in clinical applications, the adenoviruses have to be diluted to an extreme extent so as to decrease the toxicity contained in glycerine. These practices have unfavorably constrained adenoviruses from being adopted in clinical applications. Hence, to create a kind of recombinant human endostatin adenoviruses that can be stored under a temperatures in or near the range of 2° C.-8° C. for an extended period of time is an urgent and unmet need in the biopharmaceutical industry.

Lyophilizing is a technique that can be used to increase storage stability. In lyophilization processes, water can be removed from a product such as a protein, consequently enhancing the product's stability and making it more suitable for long-distance transportation and long-term storage in solid state.

As disclosed herein, lyophilized recombinant human endostatin adenovirus injection is safe, effective, stable and suitable for long-term storage and use for intravenous injections, for example, for the treatment of tumors.

### EXAMPLES

Adenoviruses applied in this invention were provided by Guangzhou Doublle Bioproducts Co., Ltd. (taking the 2^{nd} generation of recombinant adenoviruses as its vectors to carry the reconstructed human endostatin genes).

Table 1 provides an exemplary composition of the recombinant human endostatin adenovirus injection.

TABLE 1
Exemplary Composition

| | |
|---|---|
| Recombinant Human Endostatin | 10⁸-10¹² vp/mL |
| Adenoviruses | |
| Mannitol | 5.0 g-20 g |
| Sucrose | 5.0 g-20 g |
| Sodium Chloride | 100 mM-500 mM |
| Magnesium Chloride | 1.0 mM-5.0 mM |
| Tris-(HCl) Buffer Solution | 10 mM-20 mM |
| (tris(hydroxymethyl)aminomethane) | |
| Water for Injection Filled Up to 100 mL | (pH 8.0-8.8) |

Exemplary preparation procedures of lyophilized recombinant human endostatin adenovirus injections:

1. Mix each supporting ingredient (excipients) according to formulation.

2. Add purified water till it reaches 100 mL.

3. Use a 0.22-μm filter film to filter out bacteria.

4. Pour in original adenovirus fluid of recombinant human endostatin adenoviruses (1×10¹² vp/mL) in a proper ratio and mix thoroughly.

5. Pour 1 mL of the mixed solution into several 3 mL-sized glass sample bottles.

6. Place these glass sample bottles into the lyophilization chamber to start lyophilizing:
a. Place glass sample bottles onto the platform of the chamber and lower the temperature to −45° C. at a dropping rate of 1° C./min and then keep the temperature at −45° C. for 3 hours;
b. Dry the content of the sample bottles for 10 hours at −45° C.;
c. Dry the content of the sample bottles for 60 hours at −43° C.;
d. Dry the content of the sample bottles for 24 hours at 0° C.;
e. Dry the content of the sample bottles for 7 hours at 30° C.

7. Keep the vacuum pressure under 3 Pa.

8. After drying is completed, cap and label those sample bottles.

With lyophilizing as disclosed herein, the pharmaceutical compositions of the invention exhibit a number of favorable characteristics: 1) in good shape, 2) transformed into white loose powder, 3) able to absorb water in a fast manner, 4) containing no glycerine, and 5) suitable for intravenous injection. Infection titer test and reporter gene activity test have both demonstrated that the lyophilized recombinant human endostatin adenoviruses prepared according to the invention maintain the original activity of viruses. Accelerated test and long-term stability test have demonstrated satisfactory stability. The result of lyophilized human endostatin gene activity conforms with the standards stated in both Manufacture and Examination Regulations—Declaration Data 13 and Chinese Phamacopoeia Volume I/II/II.

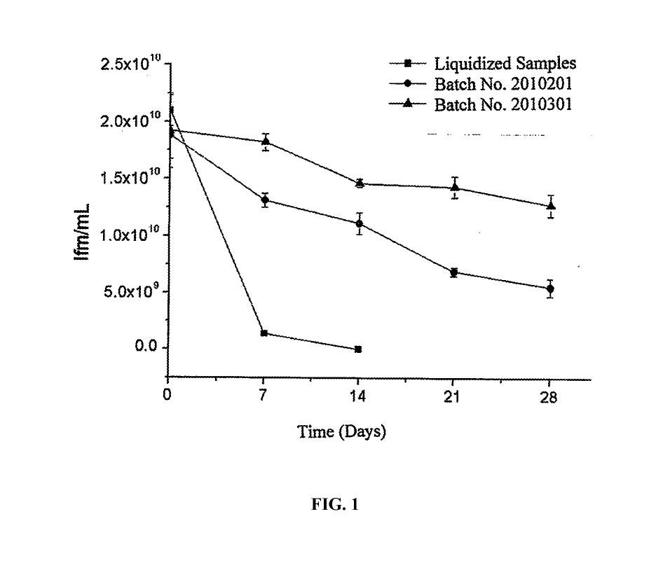

FIG. 1 shows exemplary data of stability comparison curves between lyophilized and liquidized samples under a temperature of 37° C.

### Example 1

### Preserver Prescription

Mannitol 10 g, Sucrose 10 g, Sodium Chloride1.17 g, Magnesium Chloride 0.0406 g, Tris-(HCl) buffer solution 0.242 g.

### Preparation

1. Mix each supporting ingredient (excipients) according to formulation.

2. Add purified water till it reaches 100 mL.

3. Use a 0.22-μm filter film to filter out bacteria.

4. Pour in original adenovirus fluid of recombinant human endostatin adenoviruses in a proper ratio and mix thoroughly and make sure the pH balance reaches 8.2.

5. Pour 1 mL of the mixed liquid into several 3 mL-sized glass sample bottles.

6. Place these glass sample bottles into the lyophilization chamber to start lyophilizing:
a. Place glass sample bottles onto the platform of the chamber and decrease the temperature to −45° C. at a dropping rate of 1° C./min and keep the temperature at −45° C. for 3 hours;
b. Dry the content of the sample bottles for 10 hours at −45° C.;
c. Dry the content of the sample bottles for 60 hours at −43° C.;
d. Dry the content of the sample bottles for 24 hours at 0° C.;
e. Dry the content of the sample bottles for 7 hours at 30° C.

7. Keep the vacuum pressure under 3 Pa.

8. After drying is completed, cap and label the sample bottles.

### Example 2

### Preserver Prescription

Mannitol 20 g, Sucrose 10 g, Sodium Chloride1.17 g, Magnesium Chloride 0.0406 g, Tris-(HCl) buffer solution 0.242 g.

### Preparation

1. Mix each supporting ingredient (excipients) according to formulation.

2. Add purified water till it reaches 100 mL.

3. Use a 0.22-μm filter film to filter out bacteria.

4. Pour in original adenovirus fluid of recombinant human endostatin adenoviruses in a proper ratio and then mix thoroughly and make sure the pH balance reaches 8.2.

5. Pour 1 mL of the mixed liquid into several 3 mL-sized glass sample bottles.

6. Place these glass sample bottles into the lyophilization chamber to start lyophilizing:
a. Place glass sample bottles onto the platform of the chamber and lower the temperature to −45° C. at a dropping rate of 1° C./min and keep the temperature at −45° C. for 3 hours;
b. Dry the content of the sample bottles for 10 hours at −45° C.;
c. Dry the content of the sample bottles for 60 hours at −43° C.;
d. Dry the content of the sample bottles for 24 hours at 0° C.;
e. Dry the content of the sample bottles for 7 hours at 30° C.

7. Keep the vacuum pressure under 3 Pa.

8. After drying is completed, cap and label the sample bottles.

### Example 3

### Preserver Prescription

Mannitol 10 g, Sucrose 20 g, Sodium Chloride 1.17 g, Magnesium Chloride 0.0406 g, Tris-(HCl) buffer solution 0.242 g.

### Preparation

1. Mix each supporting ingredient (excipients) according to formulation.

2. Add purified water till it reaches 100 mL.

3. Use a 0.22-μm filter film to filter out bacteria.

4. Pour in the original adenovirus fluid of recombinant human endostatin adenoviruses in a proper ratio and then mix them up thoroughly and make sure the pH balance reaches 8.2.

5. Pour 1 mL of the mixed liquid into several 3 mL-sized glass sample bottles.

6. Place these glass sample bottles into the lyophilization chamber to start lyophilizing:
a. Place glass sample bottles onto the platform of the chamber and decrease the temperature to −45° C. at a dropping rate of 1° C./min and keep the temperature at −45° C. for 3 hours;
b. Dry the content of the sample bottles for 10 hours at −45° C.;
c. Dry the content of the sample bottles for 60 hours at −43° C.;
d. Dry the content of the sample bottles for 24 hours at 0° C.;
e. Dry the content of the sample bottles for 7 hours at 30° C.

7. Keep the vacuum pressure under 3 Pa.

8. After drying is completed, cap and label the sample bottles.

### Example 4

### Titer Level Comparison Between Samples Made Before & After Lyophilization

Pour 1 mL of the recombinant human endostatin adenoviruses (containing preserver and with adenoviruses volume at a concentration level of 10¹¹ vp/mL) into each bottle. Reserve some samples for measuring the titer of the adenoviruses before lyophilization and some for making the lyophilized recombinant human endostatin adenoviruses. Measure the infection titer levels of lyophilized recombinant human endostatin adenovirus samples from 3 different batches. Measurement result: the titer level only drops around 0.2 log IFU/mL after conducing lyophilization.

TABLE 2
Recombinant Human Endostatin Adenoviruses' Titer
Level Comparison (Before vs After Lyophilization)

| | Infection Titer (log IFU/mL) | | |
|---|---|---|---|
| Batch No. | Before Lyophilizing | After Lyophilizing | Difference |
| 2010201 | 10.01 | 9.76 | 0.25 |
| 2010301 | 10.01 | 9.88 | 0.13 |
| 2010302 | 10.01 | 9.89 | 0.12 |

### Example 5

### Insect Luciferase Reporter Gene Activity Comparison Between Samples Made Before & After Lyophilization

Pour 1 mL of the recombinant human endostatin adenoviruses (containing preserver and with adenoviruses volume at a concentration level of 10¹¹ vp/mL) into each bottle. Reserve some samples for measuring the activity of the insect luciferase reporter gene carried by adenoviruses before lyophilizing and some for measuring the activity of the insect luciferase reporter gene carried by adenoviruses after lyophilizing (please refer to the user manual inside Promea E1500 Reagent box for details about how to measure the activity of insect luciferase reporter gene). Measurement result: Compared to the liquidized samples, the level of the insect luciferase reporter gene activity of the lyophilized recombinant human endostatin adenoviruses is slightly lower.

TABLE 3
Insect Luciferase Reporter Gene Activity Level
Comparison (Before vs After lyophilization)

| | Relative Activity Level of Insect |
|---|---|
| Batch No. | Luciferase (RLU/well) |
| Liquidized Sample | 398390 |
| 2010201 | 232172 |
| 2010301 | 300945 |
| 2010302 | 329814 |

### Example 6

### Recombinant Human Endostatin Reporter Gene Activity Comparison Between Samples Made Before & After Lyophilization

Pour 1 mL of the recombinant human endostatin adenoviruses (containing preserver and with adenoviruses volume at a concentration level of 10¹¹ vp/mL) into each bottle. Reserve some samples for measuring the activity of the recombinant human endostatin genes carried by adenoviruses before lyophilizing and some for measuring the activity of the recombinant human endostatin genes carried by adenoviruses after lyophilizing (please refer to the user manual inside ELISA Reagent box for details about how to test the activity of the Recombinant human endostatin genes). Measurement result: Compared to the liquidized samples, the level of activity of the lyophilized recombinant human endostatin genes is well preserved.

TABLE 4
Recombinant Human Endostatin Gene Activity Level
Comparison (Before vs After lyophilization)

| | Recombinant Human Endostatin Volume |
|---|---|
| Batch No. | (ng/mL) |
| Liquidized Samples | 56.51 |
| 2010201 | 31.40 |
| 2010301 | 34.70 |

### Example 7

### Water Content Measurement for Lyophilized Samples

Upon finishing lyophilizing the samples, take out the samples immediately and measure their water content with Karl Fischer Moisture Analysis method. Measurement result: the water content of all the samples tested is below 3%.

TABLE 5
Water Content of Lyophilized Recombinant
Human Endostatin Adenoviruses

| Batch No. | Water Content |
|---|---|
| 2010201 | 2.0% |
| 2010301 | 2.9% |
| 2010302 | 2.9% |

### Example 8

### Accelerated Experiment (at 37° C.) on Lyophilized Recombinant Human Endostatin Adenovirus Samples

Pour 1 mL of the recombinant human endostatin adenoviruses (containing preserver and with adenoviruses volume at a concentration level of 0.5×10¹² vp/mL) into each bottle. Seal some sample bottles and store them under a temperature of 37° C. without exposure to sunlight; after lyophilizing some other samples, store them under a temperature of 37° C. without exposure to sunlight. Conduct a random sampling every 7 days to measure the infection titer levels of both the liquidized and lyophilized samples. Measurement result: after 14 days of storage, the titer level of the liquidized samples drops more than 3 log IFU/mL whereas the lyophilized samples drop less than 1 log IFU/mL after 28 days of storage.

TABLE 6
Accelerated Experiment (at 37° C.) on lyophilized
recombinant human endostatin adenovirus samples

| | Infection Titer (log IFU/mL) | | | | |
|---|---|---|---|---|---|
| Batch No. | 0 day | 7 days | 14 days | 21 days | 28 days |
| Liquidized | 10.32 | 9.14 | 7.30 | ND | ND |
| Samples | | | | | |
| Lyophilized | 10.28 | 10.12 | 10.05 | 9.84 | 9.74 |
| Samples | | | | | |

In this specification and the appended claims, the singular forms “a,” “an,” and “the” include plural reference, unless the context clearly dictates otherwise.

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain important additional information, exemplification and guidance that can be adapted to the practice of this invention in its various embodiments and equivalents thereof.

What is claimed is:

1. A pharmaceutical composition comprising:
a lyophilized recombinant human endostatin adenovirus; and
a preserver composition comprising: a sugar, a polyol, an amino acid, and a salt, wherein the recombinant human endostatin adenovirus comprises a human endostatin gene.

2. The pharmaceutical composition of claim 1, wherein the preserver composition comprises:
sugar, from about 1 g to about 25 g;
polyols, from about 5 g to about 25 g;
amino acid, from about 0.1 mM to about 10 mM; and
salt, fro, about 1 mM to about 500 mM.

3. The pharmaceutical composition of claim 1, wherein the sugar is selected from one or more of: glucose, sucrose, trohalose, lactose, maltose, fructose, dextran, and inulin.

4. The pharmaceutical composition of claim 1, wherein the polylo is selected from one or more of: mannitol, sorbitol, xylitol, and isomaltitol.

5. The pharmaceutical composition of claim 1, wherein the amino acid is selected from one or more of: glycine, lysine, arginine, histidine, aspartic acid, alaline, and glutanic acid.

6. The pharmaceutical composition of claim 1, wherein the salt is selected from one or more of: sodium chloride, potassium chloride, calcium chloride, zinc chloride, magnesium chloride, citrate, tris-(HCl) buffer solution, and 4-(2-hydroxyerhyl)piperazine-1-erhanesulfonic acid.

7. The pharmaceutical composition of claim 1, wherein the adenovirus is replication-deficient recombinant adenovirus.

8. The pharmaceutical composition of claim 1, wherein each injection has a unit dosage of 10⁸ vp/mL-10¹² vp/mL of the adenovirus.

9. The pharmaceutical composition of claim 1, further comprising one or more pharmaceutically suitable excipients.

10. The pharmaceutical composition of claim 1, prepared by a method comprising:
(1) mixing one or more excipients in proper amounts and purified water resulting in a solution;
(2) filtering the resulting solution with a 0.22-μm filter to remove bacteria;
(3) adding recombinant human endostatin adenovirus while mixing and making the mixed solution a pH in a range from about 8.0 to about 8.6;
(5) lyophilizing the mixed solution;
(6) maintaining a vacuum under 3 Pa; and
(7) storing the resulting lyophilized mixture.

11. The pharmaceutical composition of claim 10, wherein the pH is about 8.2.

12. The pharmaceutical composition of claim 11, wherein lyophilizing comprises:
(a) decreasing sample temperature to about −45° C. at a rate of 1° C./min and then keeping the temperature at about −45° C. for about 3 hours;
(b) drying the sample for about 10 hours at about −45° C.;
(c) drying the sample for about 60 hours at about −43° C.;
(d) drying the sample for about 24 hours at about 0° C.; and
(e) drying the sample for about 7 hours at 30° C.

13. The pharmaceutical composition of claim 10, wherein the pH is about 8.0.

14. The pharmaceutical composition of claim 13, wherein lyophilizing comprises:
(a) decreasing sample temperature to about −35° C. at a rate of 1° C./min and then keeping the temperature at about −35° C. for about 3 hours;
(b) drying the sample for about 50 hours at about −35° C.;
(c) drying the sample for about 5 hours at about −20° C.;
(d) drying the sample for about 5 hours at about 0° C.; and
(e) drying the sample for about 2 hours at 20° C.

15. The pharmaceutical composition of claim 10, wherein the pH is about 8.6.

16. The pharmaceutical composition of claim 15, wherein lyophilizing comprises:
(a) decreasing sample temperature to about −45° C. at a rate of 1° C./min and then keeping the temperature at about −45° C. for about 3 hours;
(b) drying the sample for about 60 hours at about −45° C.;
(c) drying the sample for about 24 hours at about 0° C.; and
(d) drying the sample for about 10 hours at 20° C.
